(19) United States
(12) Patent Application Publication
(10) Pub. No.: US 2007/0068837 A1
(43) Pub. Date: Mar. 29, 2007

(54) Wound dressing dispenser

(76) Inventor: Bruno Salvatore D'Angelis, Thomastown (AU)

Correspondence Address: STEVENS & SHOWALTER LLP, 7019 CORPORATE WAY, DAYTON, OH, 45459-4238, US

(21) Appl. No.: 11/451,276

(22) Filed: Jun. 12, 2006

(30) Foreign Application Priority Data
Sep. 29, 2005 (AU) 2005905388

(51) Int. Cl. A61B 19/02

(52) U.S. Cl. 206/440

(57) ABSTRACT

A wound dressing device for dispensing individual wound dressings on demand from a roll of a multitude of identical wound dressings is described. Each individual wound dressing has a coarse open weave pad of gauze or other material containing a therapeutic agent provided with adhesive wings on the outer sides of the pad arranged in spaced end to end relationship with adjacent wound dressings sandwiched between a continuous backing strip located on one side and a continuous protective strip located on the other side. The roll of wound dressings is contained within a replaceable cassette that can be clipped onto a housing fixed to a wall of a room. The housing is provided with an operating subassembly having an operating lever. The subassembly interacts with the cassette to advance the wound dressings one at a time through the dispenser when the lever is moved to operate the subassembly. The continuous backing strips and continuous protective strips are attached to their own rollers within the cassette that are rotated to wind the strips around the respective rollers in response to movement of the lever to draw the wound dressing through the dispenser to exit through a dispensing slot provided in the cassette for removal from the dispenser. The advantage of the dispenser is that wound dressings can be dispensed more easily and more hygienically, particularly by children, since the dispenser can act as an aid to hold the wound dressing to assist in applying the wound dressing to the injured part of the body with reduced risk of the wound dressing being contaminated or sticking to itself.

### FIELD OF THE INVENTION

The present invention relates generally to dispensers for dispensing medicated wound dressings. Examples of the medicated wound dressings are sticking plasters, adhesive bandages or similar wound dressings that can be adhered in place on the skin by suitable adhesive for the treatment of minor cuts, abrasions, scratches and the like.

In one aspect, the present invention relates to a dispenser for dispensing individual medicated wound dressing products from a roll or other assemblage or array of similar wound dressings arranged in end to end relationships along a web in which the medicated wound dressings are of the type having a protective strip over a layer of adhesive of the sticking plaster for adhering the wound dressing to the skin. The would dressing is applied over the wound by being applied to the skin in or at the region around or in close proximity to the peripheral portions of the wound so that a medicated pad or similar can cover the wound either partially or wholly. The wound dressing can be applied to the wound when the sticking plaster is removed from a backing strip or web of the wound dressing by removing both the protective strip and the backing layer or strip from the sticking plaster.

The present invention finds particular application as a dispenser for dispensing individual medicated products from a strip of a multitude of similar products interconnected together when stored within the dispenser in the form of a roll in response to activation of the dispenser, such as by manually moving a lever or similar actuating element to advance the roll through the dispenser to dispense individual pads separate from the adhesive protective layer and/or backing layer or strip which are removed from the sticking plaster when the sticking plaster is being dispensed.

Although the present invention will be described with particular reference to one form of the dispenser for dispensing one form of the wound dressing products having a backing strip, a medicated pad and a protective layer, it is to be noted that the scope of the present invention is not restricted to the described embodiments but rather the scope of the present invention is more extensive so as to include other forms and arrangements of the dispenser, other forms and arrangements of the wound dressing, the use of the various forms of the dispenser for dispensing products other than the particular medicated wound dressing described and the use of the dispenser in other applications.

### BACKGROUND OF THE INVENTION

A sticking plaster is generally a small patch of coarse bandage, set in a strip of flesh-coloured adhesive tape. It is used to cover cuts and wounds that are small enough not to require stitches or surgery such as minor cuts, abrasions, scratches and the like. Sticking plasters are known under other names such as adhesive bandage and are one form of wound dressings. A sticking plaster usually consists of an absorbent pad (often medicated with antiseptic) covered by a layer of woven fabric or a plastic adhered to a tape which is covered in a layer of adhesive, particularly on either side of the medicated pad. The plaster is applied to the skin such that the pad covers the wound but does not stick to the wound and the fabric or plastic sticks to the surrounding skin by the adhesive to hold the dressing in place spanning over the wound to prevent dirt and other contaminants from entering the wound. Sometimes the word medical dressing is used to refer to sticking plaster. Two well known brands of sticking plasters are Elastoplast™ and Band Aid™. In one embodiment, the dispenser of the present invention is designed to dispense individual “Band Aids™” from a roll of “Band Aids™”.

Sticking plasters or adhesive bandages usually are provided as a single sterilised packet or sachet which remains in a sealed condition until it is required for use. When required for use the sealed sachet is torn open, the plaster withdrawn from the package, the protective sheet removed from the adhesive layer on one side of the sticking plaster to expose the adhesive layer and the adhesive of the sticking plaster applied to the skin of a person close to or around at least a part of the peripheral portion of the wound to adhere to the skin so as to cover the wound thereby providing protection for the wound as well as treating the wound with a medicated material contained within the pad to assist in healing of the wound.

Often, it is inconvenient for a person suffering from a cut, such as a for example on the finger, to manipulate the sterilised sachet to open it, peel the protective cover over the adhesive layer from the sticking plaster and apply the bandage to the wound, particularly without the adhesive portion sticking to itself or to another part of the plaster. Thus, there is a need to provide a dispenser for a sticking plaster wound dressing that allows the wound dressing to be more easily dispensed and moreover, allows the sticking plaster to be dispensed in a condition allowing it to be applied to the skin more conveniently.

Often, the greatest consumers of sticking plasters are small children. However, small children usually do not have the manual dexterity to manipulate the sterile packages and to open them or remove the protective strip from the adhesive layer without the adhesive contacting unwanted surfaces which reduces the effectiveness of the sticking plaster. Thus, there is a need for a dispenser that can be operated by children, particularly small children, that makes sticking plasters available in a condition ready for use so that all the child needs to do is apply the adhesive bandage to their skin about the wound after it is removed from the dispenser or while the bandage is still attached to the dispenser but in an exposed state.

Additionally, there is a cost associated with providing individual sterilised sachets or packets, each containing a single bandage only. If it were possible to provide an arrangement of sticking plasters that did not require individual sterilised packets that needed to be opened one at a time, cost savings could be obtained in not having to manufacture individual sterilised packages. Thus, there is a need for a dispenser that allows individual plasters to be made available for use by dispensing the sticking plasters one at a time without having to open individual sterilised packets or sachets.

Thus, there is a need to provide a dispenser for sticking plasters that is easier to use to dispense the sticking plasters, and there is a need for a dispenser that allows easier separation of the protective strip covering the adhesive layer from the sticking plaster itself. Additionally, there is a need for a method of making and dispensing sticking plasters that involves less expense to manufacture the sticking plasters.

According to one aim of the present invention there is provided a dispenser for dispensing a sticking plaster in a condition ready for use in which the backing sheet and/or protective cover strip have been removed from or are more readily removable from the sticking plaster allowing the sticking plaster to be applied to the wound.

### SUMMARY OF THE INVENTION

According to one aspect of the present invention there is provided a dispenser capable of dispensing one item from a multitude of similar items arranged so that one part of the supply of items is connected to a moveable member which is moveable in response to movement of an actuating element such that movement of the actuating element dispensers the item from the dispenser by moving the item.

According to another aspect of the present invention there is provided a dispenser capable of dispensing individual articles from an array or a web of similar articles, said articles including at least one removable layer and a medicated element, said dispenser comprising a storage member for storing the array or web of similar articles, an actuating element and a movable member associated with the web or array of articles arranged so that movement of the actuating element causes movement of the movable member to advance at least one of the similar articles or array or web through the dispenser and to separate the at least one removable layer from the medicated element so that the article can be dispensed from the dispenser in a condition ready for use.

According to another aspect of the present invention there is provided a method of dispensing an article from a multiplicity of similar articles, each article including at least one removable layer adhered to the article, comprising the steps of moving an actuating element to advance at least one of the multiplicity of articles through the dispenser so that the removable layer is separated from the article simultaneously with or prior to dispensing of the article from the dispenser so that the article is in a condition for use.

According to one aspect of the present invention there is provided a dispenser capable of dispensing a medicated item from a multitude of substantially similar medicated items, each of said items having at least one removable layer attached to the medicated items when in the dispenser, said at least part of one removable layer connected to or in contact with a movable member arranged so that movement of an actuating element produces corresponding movement of the moveable member to advance at least one of the medicated items through the dispenser, said removable layer being arranged to move through the dispenser so that as the medicated item advances towards a dispensing slot provided in the wall of the dispenser the at least one removable layer is separated from the medicated item to allow the medicated item to be discharged from the container through the slot separated from the at least one layer which remains in the dispenser so that the medicated item is in a condition ready for use.

### BRIEF DESCRIPTION OF THE INVENTION

Typically, the item is a healing item, a therapeutic item, a medicated item or similar for providing treatment to a person to whom the item is applied, typically a topical treatment. More typically, the medicated item is a wound dressing or part of a wound dressing. More typically, the wound dressing has a medicated or therapeutic part of portion, preferably a pad or the like for contracting the skin or wound in the surface of the skin. More typically, the wound dressing is a sticking plaster or adhesive bandage or similar. More typically, the wound dressing is in the form of a sandwich type construction or assemblage having at least three layers in which the sticking plaster is one layer which is located intermediate a second layer being a removable protective cover strip for protecting and/or covering the adhesive portion of the sticking plaster and/or adhesive provided on the sticking plaster and a third layer being a backing strip or web of material on which the sticking plaster is located or mounted so as to form a roll or array of sticking plasters. Alternatively, the wound dressing can include two layers only being the protective strip and sticking plaster only or a backing strip and sticking plaster only i.e. a double layer construction rather than a triple layer construction. Other constructions of the medicated wound dressing are also possible, including having different numbers of layers, different forms, different arrangements and the like.

Typically, the wound dressing is in the form of an elongate strip having a large number of individual sticking plasters located in end to end relationship along the strip typically in abutting end to end relationship or in spaced apart end to end relationship or similar. Alternatively, the sticking plasters can be arranged in side by side relationship or in any other spatial relationship to each other or combinations of spatial relationships or the like. More typically, the individual sticking plasters can be spaced apart from each other or in abutting relationship either in side by side abutting relationship or end to end abutting relationship. Additionally, in some forms, the sticking plasters can be arranged in stacked relationship being located one above the other. Even more typically, the elongate strip is in the form of a roll having up to about 500 or more individual sticking plasters, preferably up to about 250 sticking plasters, more preferably from about 50 to 100 individual sticking plasters on the one roll located in end to end relationship. Alternatively, the multitude of sticking plasters can be arranged in looped turns located in either side by side or on top of each other to form a continuous convoluted array of sticking plasters folded back upon themselves.

Typically, the items to be dispensed are single items or individual items or are in groups of items or the like. The single items can be dispensed singly as individual items or collectively as groups of two or more individual items. More typically, the items can be dispensed in any order, arrangement or in any number.

Typically, the individual sticking plasters are located at regularly spaced locations along at least a portion of the length of the elongated strip. More typically, there is a line of weakness between individual sticking plasters, such as a perforated line or similar making it easier to separate individual plasters from each other and hence from the roll, array, stack or the like, particularly when dispensing the sticking plasters. More typically, the sticking plasters are arranged in segments, sections, groups or the like. Even more typically, the sticking plasters are easily separable from each other or from the roll by some arrangement such as for example the arrangements used to separate individual plastic bags from a roll of plastic bags having a seal line of one bag in close proximity to the open end of an adjacent bag.

Typically, the sticking plasters are continuous and the dispenser is provided with a cutter. Typically, the cutter is in the form of a blade or similar, for cutting the size of the sticking plaster to length in accordance with the needs for the sticking plaster, particularly immediately prior to or at the time of dispensing the sticking plaster. More typically, the action of the cutter is linked to the movement of the dispenser so that the roll of sticking plasters is cut when part of the roll is dispensed or on dispensing part of the roll or the like or as the sticking plaster advances through the dispenser prior to cutting the web. More typically, there is simultaneous cutting and dispensing of the sticking plasters. In some embodiments, the sticking plaster is cut, but that the backing strip remains continuous, whereas in other embodiments the protective strip and sticking plaster are cut. In some embodiments, the operation of the cutter is linked to operation of the actuating lever whereas in other embodiments, operation of the cutter is independent of the movement of the actuating element or lever.

Typically, the dispenser is manually operable or automatically operable by having a motor or the like, such as an electrically operable servo motor, for advancing the wound dressing through the dispenser in response to operation of a control such as for example, a switch. More typically, the dispenser is mains powered or is battery powered, such as by a rechargeable battery or similar provided within or connected to the dispenser. More typically, the dispenser is made from plastics material. Even more typically, the dispenser includes a sterilising agent or a sterilising device for maintaining the wound dressings in a sterilised condition when in the dispenser. Typically, the dispenser or roll of sticking plaster or the like is in a sterile package or similar. More typically, the dispenser includes a swipe of sterilising agent for wiping the wound dressing as it moves through the dispenser to sterilise or maintain sterile, the array of wound dressings or individual wound dressings.

Typically, the dispenser includes a subassembly for controlling operation and movement of the wound dressing. The subassembly can take a number of different forms or arrangements, and can include other devices for effecting movement of the wound dressing through the dispenser including other devices for contacting the wound dressing directly or indirectly to advance the wound dressing through the dispenser.

Typically, the dispenser is a cassette or comprises a cassette and housing or body, more typically a cassette which is replaceably insertable within a cavity or chamber within the dispenser, housing or body or is replaceably receivable in a holder or cradle for securing the cassette in place. More typically, the cassette is a removable and replaceable cassette for exchanging when empty with a another cassette having a fresh full roll of sticking plasters or similar, by removing the spent cassette from the holder, cradle, chamber or the like and inserting the full cassette in its place in the holder, cradle, chamber or the like. Typically, the cassettes can have the same or different numbers of sticking plasters. Typically, the dispenser includes a housing or a removable cover for one or more of the chambers, more typically, for two, three, four or more chambers.

More typically, the dispenser includes a cassette and holder in which the cassette has the roll of sticking plasters and the holder is fixed to a suitable substrate such as the wall of a room or similar for forming a cradle for the cassette. More typically, the holder includes the operating subassembly. More typically, the cassette is received within or by the holder as the cassette is clicked into place onto the holder. More typically, the cassette is snap fittingly received in the holder. Even more typically, the holder is provided with lugs or similar for cooperatively engaging with apertures provided on the cassette to retain the cassette in place on or in the holder.

Typically, the dispenser or cassette is provided with a chamber for containing the roll of layered wound dressing or a stack of individual wound dressings arranged in a suitable array, including a looped array. More typically, the dispenser or cassette has one or more other chambers for receiving parts of the layered wound dressing after the sticking plaster has been dispensed, such as the waste removable layer when separated from the wound dressing, the protective layer when removed and separated from the wound dressing or the like. Typically, the waste layers are discarded when the empty cassette is disposed of.

Even more typically, the individual layers of the layered wound dressing are separated from the sticking plaster simultaneously with dispensing of the sticking plaster.

Typically, the subassembly of the dispenser is located within the holder and is provided with an actuating element. More typically, the actuating element is a lever or other moveable element such as a thumb wheel, push button, pull cord, or other manually operable element.

More typically, the actuating lever extends from the subassembly or holder and is accessible from outside the holder or subassembly.

More typically, the actuating element is connected to the movable member. More typically, the moveable member is a roller, wheel, cylinder, shaft, a former or other rotatable member. More typically, the moveable member can take any profile or cross-sectional shape such as for example, being circular, elliptical, hexagonal, octagonal, square or other polygonal shape or the like. More typically, the moveable member is located centrally within the roll of wound dressings so that movement of the moveable member rotates the roll of wound dressings.

### BRIEF DESCRIPTION OF THE DRAWINGS

The present invention will now be described by way of non limiting example with reference to the accompanying drawings in which:

FIG. 1 is a front side perspective view of one form of the internal arrangement of one form of the dispenser of the present invention with the front cover removed to reveal the internal storage chambers of the dispenser containing individual rolls.

FIG. 2 is a front elevation view of the dispenser of FIG. 1 with the front cover removed.

FIG. 3 is a partially exploded perspective view of the dispenser of FIG. 1 showing the operating sub assembly located at the rear of the dispenser for rotating the rolls during use of the dispenser.

FIG. 4 is a front elevation view of the form of the operating sub assembly as shown in FIG. 3.

FIG. 5 is a longitudinal cross-section of the dispenser of the present invention showing the dispenser located in situ attached to a wall in an in use condition showing a side view of the storage chambers and the operating mechanism.

FIG. 6 is a front elevation view of a modified form of the dispenser.

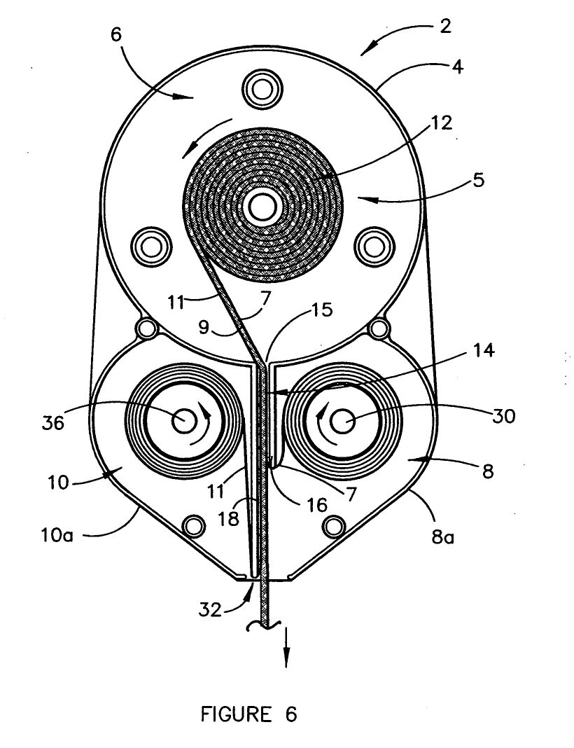

FIG. 7 is a front elevation view of a modified form of the operating subassembly.

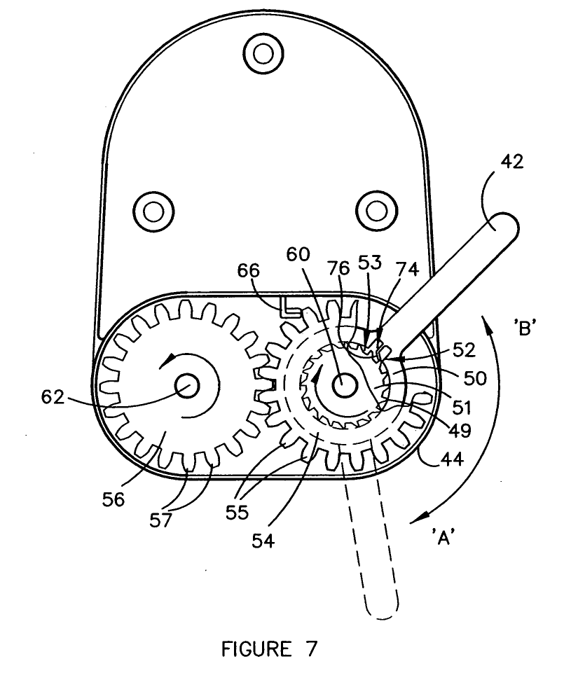

### DETAILED DESCRIPTION OF THE PREFERRED EMBODIMENTS OF THE INVENTION

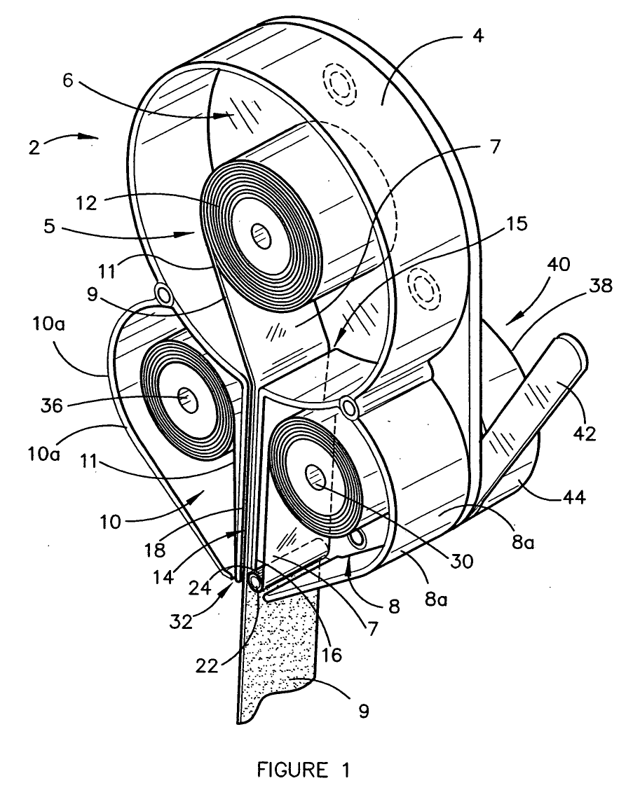

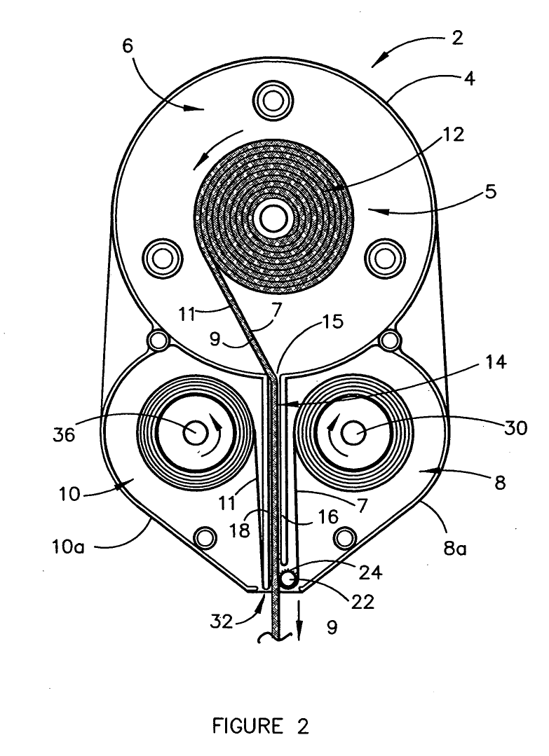

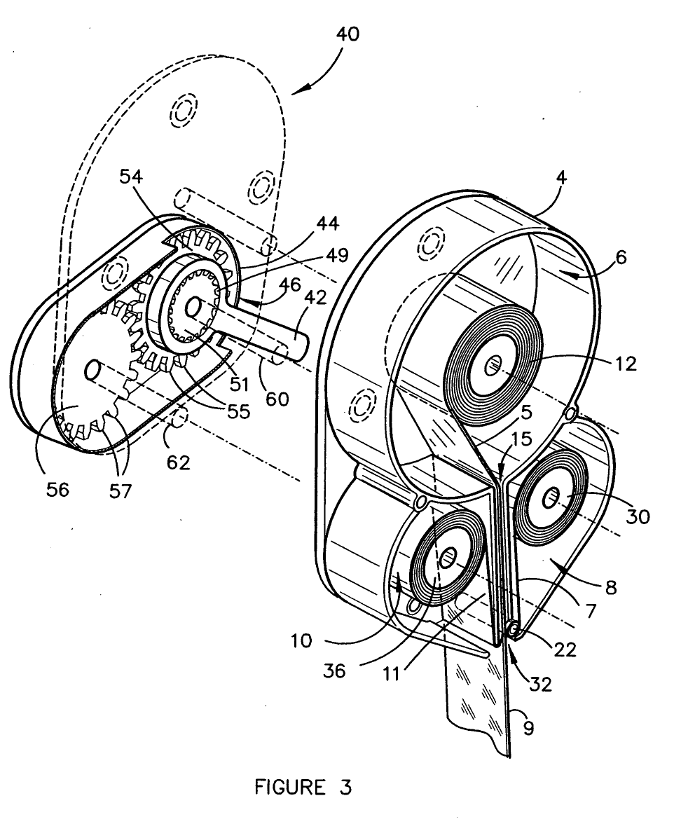

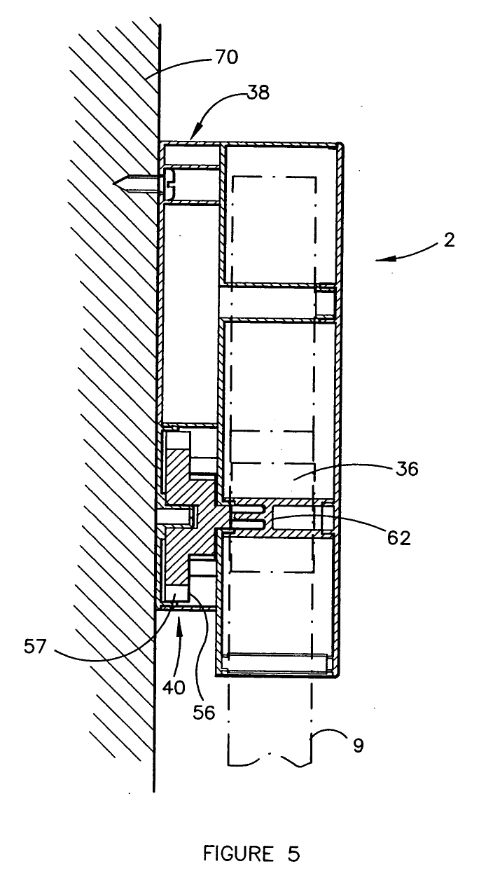

In the drawings there is shown one form of the dispenser of the present invention. This form of the dispenser is a two part dispenser which has a cassette or housing 2 clicked into place on a holder 38 which is fitted to a suitable substrate. Thus, the cassette is accessible at the front of the holder. Other forms of the dispenser are possible as is the form of the housing or similar for containing the cassette, and the cassette itself. The cassette which contains the roll of wound dressings is usually a sealed unit and is replaced as a single unit. Alternatively, the dispenser is provided with a removable front cover allowing access to the inside of the housing within which the cassette is located. In one embodiment, cassette or housing 2 is in the form of a generally curved body having a number of separate compartments. One of the compartments is a generally circular shaped main storage chamber 6 located on a generally upper portion of the cassette when in the in use position attached to a suitable substrate as shown in FIG. 5. Chamber 6 is defined by curved wall 4 around the upper periphery and is for storing a roll 12 of a web of sticking plasters 5. The other compartments are two generally curved-sided waste-receiving chambers 8, 10 located lower than and to either side of main chamber 6. Waste receiving chambers 8, 10 are defined around their respective outboard sides by curved walls 8a and 10a, respectively. Chamber 8 is for receiving the backing strip of the sticking plaster when removed from the sticking plaster and chamber 10 is for receiving the protective strip when removed from the sticking plaster. Normally, the front of the dispenser or of the cassette or housing 2 would be covered with a front cover, particularly if the cassette is a sealed unit. However, the cover has been omitted from the drawings to reveal the internal details of the dispenser or cassette as shown in FIGS. 1 to 3 of the drawings as an aid to understanding the present invention more fully. Since the cassette containing the wound dressing is removable from the holder, the cassette can be easily and quickly removed from the holder and another cassette clicked into place thereby allowing the quick exchange of cassettes of fresh rolls of sticking plaster when the supply of existing sticking plasters has been exhausted. Storage chamber 6 of the cassette is of a generally circular shape for receiving a generally cylindrical roll 12 of wound dressings including a multitude of adhesive plasters located in end-to-end generally regularly spaced apart relationship over almost the entire length of a backing strip rolled upon itself to form the roll of sticking plasters.

In one form the roll 12 of wound dressing 5 includes a sandwich construction of a backing strip or web 7, a sticking plaster 9 and a protective strip 11 in which the sticking plaster is located between the backing strip 7 on one side and the protective strip 11 on the other side. The roll 12 is formed by winding through multiple turns an elongate length of wound dressing 5 comprising the backing material 7, the sticking plaster 9, and protective layer 11 onto a suitable former located at the centre of roll 12 to form the roll 12. A suitable former could be a roller, cylinder or the like. The sticking plaster is in the form of an antiseptic coarse cotton pad or similar having a therapeutic material for treating the wound adhered onto a plastic strip extending from either side of the pad in the form of wings. The wings have applied to them an adhesive layer on one side for adhering to the skin of a person in the region of the wound. The sticking plaster is located on the backing strip or web, such as by heat sealing, an adhesive or similar. The sticking plaster is sandwiched between the backing strip 7 and the protective covering strip 11 covering the adhesive layer on the wings of the sticking plaster. The protective covering 11 is adhered on to the backing strip along their respective sides by a suitable releasable adhesive that maintains the protective strip in contact with the backing strip to protect the sticking plaster yet allows the protective strip to be readily released from the sticking plaster and backing strip when required, such as on dispensing the sticking plaster. An opening 15 is provided in the lower portion of circular wall 4 of storage chamber 6. Opening 15 forms the entrance to an elongated channel 14 formed from two spaced apart straight walls 16, 18 extending from the curved wall 4 of storage chamber 6 in opposed facing relationship to each other to form channel 14 therebetween. Channel 14 is for receiving part of roll 12 containing the wound dressing 5 in preparation for dispensing the sticking plaster 9.

It is to be noted that the length of wall 16 forming one side of channel 14 is different to the length of the other side wall 18 forming the other side of channel 14 to allow dispensing of the wound dressing as will be described in more detail. In one embodiment, wall 18 extends from the wall 4 of chamber 6 to the end of wall 10a of chamber 10 and wall 16 extends from wall 4 of chamber 6 to only part way along wall 18, typically about to the mid-point of wall 18. In another embodiment, wall 16 extends to almost the same length as wall 18. Walls 16, 18 can be of any length and any relationship to each other.

In one embodiment, a freely rotating roller 22 having a corrugated outer surface 24, such as for example, including a multitude of teeth or projections extending radially outwards, is located at or towards the end of wall 16 for directing and conveying the backing strip 7 of the sandwich construction of the wound dressing 5 into chamber 8 for receiving waste backing strip once removed from the wound dressing 5 by being wound around hollow roller 30 or similar located centrally within chamber 8.

In other embodiments, roller 22 can be omitted. If roller 22 is omitted, wall 16 extends only about half the length of wall 18 so as to leave a large gap or space between the respective ends of walls 16, 18 to allow the backing strip to be removed from the wound dressing to be received in chamber 8.

The longer sidewall 18 forming channel 14 terminates at or next to dispensing slot 32 provided in the lower portion of the side walls of cassette 2 in the region where curved side walls 8a and 10 a terminate between the backing strip waste chamber 8 and protective strip waste chamber 10 located generally symmetrically on either side of channel 14. The distil ends of walls 16, 18 are curved or rounded so as to act as a guide for the waste strips being removed from the wound dressings. The protective strip 11 is peeled from the sandwich assembly of the wound dressing 5 as it passes around the end of wall 18 as it is wound around hollow roller extension 36, located centrally within chamber 10 for receiving the waste protective strip, as roller 36 rotates. Similarly, the backing strip 7 is separated from wound dressing 5 as it passes around the end of wall 16 as it is wound around roller 30 which is located centrally within chamber 8 for receiving waste backing strip, as roller 30 rotates. The sticking plaster 9 is dispensed through dispensing slot 32 after the protective layer 7 and backing layer 11 have been removed from the respective sides of sticking plaster 9.

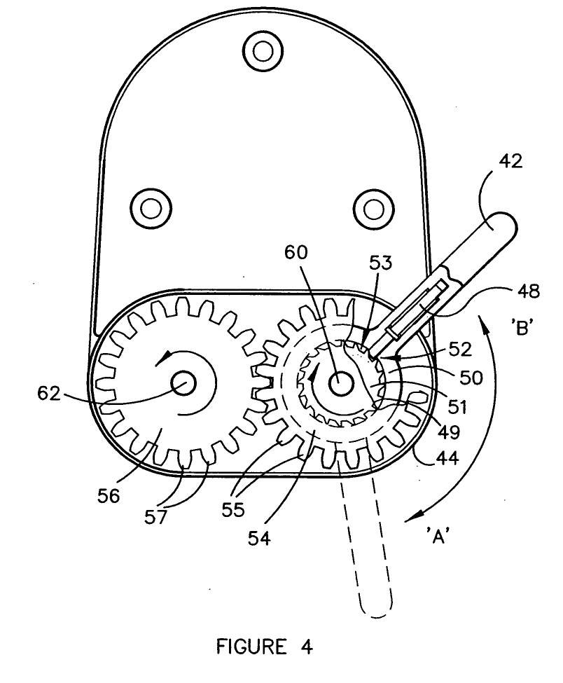

Holder 38 houses operating subassembly 40. Operating sub assembly, generally denoted as 40, is provided on the rear surface of the dispenser as shown more particularly in FIGS. 1, 3 and 5. Operation of sub assembly 40 advances the strip of wound dressings 5 through the dispenser 2 to dispense each sticking plaster in turn from dispenser slot 32. Sub assembly 40 will now be described in detail. In one form the operating sub assembly 40 includes an actuating element in the form of an elongate operating lever 42 extending radially outwardly from one side of generally oval shaped housing 44 provided with slot or cut out 46 extending around one curved side of housing 44. It is to be noted that other forms of the actuating element are possible, such as for example, a thumbwheel, a pull cord or similar. In one embodiment, a movable pin 48 is axially aligned along the lengthwise extending axis of elongate lever 46 for axial movement between an extended position as show in FIG. 4 and a retracted position (not shown). The proximal end of lever 42 is provided with a ring 50 for surrounding a ratchet arrangement 52 comprising a toothed wheel 51 having a multitude of grooves 53 located between adjacent teeth 49 into which the end of pin 48 can be received selectively. FIG. 4 illustrates the end of pin 48 being received in one of the grooves. Toothed wheel 51 is fixedly connected to gear wheel 54 or is integral with gear wheel 54 so that as wheel 51 is caused to rotate, so also is gear wheel 54. Pin 48 and ring 50 cooperatively engage with the toothed wheel 51 of the ratchet arrangement 52 to rotate the gear wheel 54 in one direction only which is the clockwise direction as shown in FIG. 4 by arrowhead A. When pin 48 is in the extended position the end of the pin engages one of grooves 53 of the ratchet arrangement so that as lever 42 is moved in the direction of arrowhead ‘A’ first toothed wheel 54 rotates as shown in FIG. 4. When lever 42 is in its lower or lowermost position corresponding to the full extent of rotation of lever 42 in the clockwise direction as shown in phantom in FIG. 4, pin 48 is moved axially outwardly along lever 42 so as to be disengaged from one of grooves 53 of the ratchet arrangement 52 thereby releasing lever 42 and ring 50 from ratchet arrangement 52 which allows lever 42 to return to the upper position in an upstroke movement by moving in an anti-clockwise direction as shown by arrow head B of FIG. 4 without moving gear wheel 54 so that the lever can return to its original position whilst the first gear wheel 54 remains stationary.

In another form of operating subassembly 40, pin 48 is replaced by a spigot 74 or similar protrusion extending into the interior of ring 50 for allowing one way rotation of gear wheel 54. In one form, spigot 74 is in the form of a generally bent arm 76 for engaging the teeth 49 of gear wheel 51 to allow rotation in one direction only which is the clockwise direction. Rotation in the anticlockwise direction is prevented since the distal end of bent arm 74 does not and cannot engage teeth 49 of gear wheel 51. Operation of lever 42 is the same when spigot 74 is present.

A lug 66 having a general L-shape is provided near to the circle prescribed by teeth 55 of gear wheel 54 as it rotates in an anticlockwise direction to allow clockwise rotation of wheel 54 but to prevent anti-clockwise rotation of gear wheel 54 by engaging against the teeth 55 of gear wheel 54 thus allowing one way rotation of the operating subassembly.

A second toothed gear wheel 56, is arranged to meshingly cooperate with the first toothed gear wheel 54, by the teeth 55 of gear wheel 54 cooperatively engaging with teeth 57 of gear wheel 54 so that rotation of the first gear wheel 54 in a clockwise direction causes corresponding rotation of the second gear wheel 56 in the opposite direction which is the anticlockwise direction as shown in FIG. 4. Therefore operation of level 42 causes the first toothed gear wheel 54 and the second tooth gear wheel 56 to rotate in unison in opposite directions to the same extent.

A first moveable member in the form of a first roller 60 which is associated with the first gear wheel 54 is provided to extend into backing strip waste receiving chamber 8. In one embodiment, first roller 60 is received within hollow roller 30 so that as roller 60 is caused to rotate so also is hollow roller 30 which in turn winds backing strip 7 onto roller 30. It is to be noted that roller 60 can have an octagonal shape or cross-section. A second roller 62 is associated with the second gear wheel 56 and extends into protective cover strip waste receiving chamber 10. In one embodiment, outwardly extending roller 62 which is of octagonal cross-section, is received within hollow roller 36 so that as roller 62 is caused to rotate, so also is roller 36 caused to rotate. It is to be noted that the moveable member can take any other suitable form or arrangement for winding the backing strip and/or the protective strip onto a roll or similar.

Operation of one form of the dispenser of the present invention will now be described.

A roll 12 of wound dressings 5 having a sandwich construction of backing sheet 7, medicated pad with adhesive layer 9 and protective covering 11 is located within storage chamber 6 of dispenser 2 with the free end of the roll 12 located extending through opening 15 and channel 14 so as to extend outwardly from dispensing slot 32 located in the lower edge in use of the dispenser when attached to a suitable substrate such as a wall 70 as shown in FIG. 5. Additionally, the free end of the protective cover strip 11 is peeled away from the sandwich construction and wound around roller 36 which in turn is associated with the roller 62 and second toothed wheel 56 so that as the toothed wheel 56 is caused to rotate in an anticlockwise direction so also is roller 36 caused to rotate in an anticlockwise direction so that the protective strip is wound in a roll around the roller 36.

Also, the free end of the backing strip 7 of wound dressing 5 is threaded over the outer surface of freely rotating roller 22, when provided, having corrugated outer surface 24 and fixedly connected to or around roller 30 which is associated with roller 60 and first toothed wheel 54 so that the backing strip 7 is able to be wound onto the roller 30 when roller 60 and toothed wheel 54 rotates in a clockwise direction. The position of the protective cover strip 11 and backing strip 7 is adjusted so that one sticking plaster is located at or within or extending from the dispenser slot 32 of the dispenser. Thus, the dispenser is ready for use.

When a sticking plaster is to be dispensed, pin 48 or spigot 72 is moved to the extended position so as to engage teeth 49 of gear wheel 57 forming ratchet 52 by being received in grooves 53 of toothed wheel 51 and actuating lever 42 is moved in a clockwise direction from the upper position to the lower position in a downstroke movement in the direction of arrowhead ‘A’ of FIG. 4 thereby rotating toothed wheel 51 and the first gear wheel 54 in a clockwise direction and correspondingly rotating the second gear wheel 56 in a anticlockwise direction so as to draw the strip of sandwich construction of wound dressing 5 including the sticking plaster 9 through channel 14 and to separate the backing strip 7 from the sandwich construction and also to separate simultaneously the protective strip 11 from the sandwich construction to expose the sticking plaster 9 through dispensing slot 32 by winding the backing strip 7 and protective strip 11 onto the respective rollers 30, 36. It is to be noted that both the backing strip 7 and protective strip 11 are forced to move by being wound onto the respective rollers 30, 36. It is the movement of the backing strip and protective strip that causes the sticking plaster 9 to move.

Pin 48 is then retracted to be free of engagement from grooves 53 of ratchet arrangement 52 or arm 76 of spigot 74 is released from teeth 49 of gear wheel 51. Actuating lever 42 can then be allowed to return from the lower position to the upper position in an upstroke movement in an anticlockwise direction so that the lever 42 can return to its original position in the upper position ready for further operation. Owing to the pin 48 at the end of the lever 42 being disengaged from groove 53 and ratchet arrangement 52 or arm 62 being released from teeth 49, lever 42 is free to move from the lower position to the upper position without causing corresponding movement of the first toothed gear wheel 54 which remains stationary.

If necessary, pin 48 can be moved to re-engage the ratchet arrangement 52 so that the actuating lever 42 can be moved again from the upper position to the lower position to further rotate the first toothed wheel 54 thereby winding more of the backing strip around roller 30 to further advance the sticking plaster and simultaneously to wind the protective cover strip 11 around roller 36 associated with the second toothed gear wheel 56 thereby exposing more of the sticking plaster further through discharge slot 32 allowing the sticking plaster to be readily removed from the dispenser and applied to the wound, even by a small child. It is to be noted that it is not necessary to move lever 42 to its full extent on each downstroke stroke to dispense an individual sticking plaster. A partial downstroke is also possible depending upon the exact position of the sticking plaster being dispensed and the part of the body to which the sticking plaster is to be attached. Furthermore, the sticking plaster can be partially attached to the body before being dispensed fully from the dispenser if required.

Thus, using the dispenser of the present invention it is possible to dispense a single sticking plaster at a time hygienically by not having to touch the sticking plaster prior to removing it from the dispenser and also using the lever allows the required amount of sticking plaster to be advanced through the dispensing slot.

When the roll of sticking plasters in one cassette is exhausted, the cassette containing the waste protective strip and waste backing strip can be removed from holder 38 and discarded thereby also discarding the waste strips. A new cassette containing a fresh roll of sticking plasters can be clicked into place on the holder ready for dispensing more sticking plasters.

### ADVANTAGES OF THE INVENTION

Advantages of the present invention include a dispenser having the capacity to hold a very large number of individual sticking plasters in a web arrangement or in a stacked arrangement or similar array of sticking plasters. Individual sticking plasters are held in a sterile and hygienic condition prior to being dispensed from the dispenser. The individual sticking plasters are dispensed without having to be touched by human hands by using a remote lever. In some embodiments, there is also no need for each sticking plaster to have its own sterilised package. Having a roll of individual sticking plasters located on a backing webbing or similar is less expensive to produce than individual sterile packages or sachets containing a single wound dressing only.

Also the sticking plaster being dispensed is held in the dispenser which acts like a “third hand” to hold the sticking plaster which makes it easier to apply the sticking plaster to inconvenient places on the body, such as the fingers or the like to which access is difficult.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be understood to persons skilled in the art of the invention that many modifications may be made without departing from the spirit and scope of the invention.

1. A dispenser capable of dispensing a single item from a multitude of similar items comprising a housing for containing the multitude of similar items, the item having a first part and a second part, said second part being separable from the first part, said plurality of items being arranged so that the second part is connected to a movable element provided within the housing, said movable element being movable in response to movement of an actuating member such that movement of the actuating member moves the movable element to dispense from the dispenser at least one first part of one item from the multitude of similar items and to separate the second part of the at least one item from the first part of the at least one item.

2. A dispenser according to claim 1 in which the multitude of similar items are arranged in an array or a web, and the first part of the item includes a therapeutic element and a second part is a removable layer, said dispenser comprising a storage compartment for storing the array or web of similar articles within the housing, the actuating element for causing operation of the dispenser to dispense one of the multitude of items and the movable member for moving at least a part of the item such that the movable member moves in accordance with movement of the actuating element so that when the actuating element is moved, the movable member is caused to move to advance at least one of the multitude of similar articles through the dispenser and to separate the at least one removable layer from the therapeutic element so that the article can be dispensed from the dispenser in a condition ready for use.

3. A dispenser according to claim 2, in which each of said items has one or two removable layers attached to the medicated item when in the dispenser, wherein said one or two removable layers are connected to one or more movable members arranged so that movement of an actuating element produces corresponding movement of the movable member or movable members to advance at least one of the medicated items through the dispenser, said removable layer or layers being arranged to move through the dispenser so that, as the medicated item advances towards a dispensing slot provided in the wall of the dispenser, the one or two removable layers are separated from the medicated item to allow the medicated item to be discharged from the container through a slot separate from the one or two removable layers which remain within the dispenser.

4. A dispenser according to claim 2 in which the item is a medicated wound dressing in the form of a sticking plaster having one or more adhesive portions in which there is a protective layer applied over the adhesive portion or portions.

5. A dispenser according to claim 4 in which the wound dressing is in the form of a sandwich type construction having three layers in which the sticking plaster is one layer which is located intermediate a second layer being the removable protective cover strip for protecting or covering the adhesive portion of the sticking plaster and a third layer being a backing strip or web of material on which the sticking plaster is located or mounted.

6. A dispenser according to claim 5 in which the wound dressing is in the form of an elongate strip having a multitude of individual sticking plasters located in end-to-end relationship along the strip.

7. A dispenser according to claim 1 further comprising a cutter for cutting the individual items from the multitude of similar items.

8. A dispenser according to claim 1 in which the dispenser is manually operable or is automatically operable by having a motor including an electrically operable motor.

9. A dispenser according to claim 1 in which the multitude of similar items are stored within the housing of the dispenser in the form of a roll of items in which the multitude of items are arranged in end to end relationship.

10. A dispenser according to claim 1 in which the actuating element is a lever, thumbwheel, pull cord or the like.

11. A dispenser according to claim 1 in which the movable element is a roller, wheel, tube, cylinder, shaft or the like.

12. A dispenser according to claim 1 in which the housing is provided with a chamber for receiving a roll of sticking plasters or wound dressings.

13. A dispenser according to claim 5 in which there are two counter rotating rollers operable to rotate simultaneously in opposite directions in response to movement of the actuating element to remove the backing strip and protective strip from the wound dressing.

14. A dispenser according to claim 12 in which there are three chambers comprising a first chamber for receiving a roll of wound dressings, a second chamber for receiving the backing strip when removed from the wound dressing and a third chamber for receiving the protective cover when removed from the wound dressing.

15. A dispenser according to claim 1 in which the dispenser is provided with a slot and channel arrangement through which the sticking plaster is dispensed.

16. A dispenser according to claim 15 in which the entrance to the slot and channel arrangement for receiving the wound dressing prior to the wound dressing being dispensed from the dispenser is located in the wall of the first chamber and the walls defining the channel are the walls of the second and third chambers.

17. A dispenser according to claim 16 in which the exit of the slot and channel arrangement is located at the discharge end of the channel formed between the second and third chambers for dispensing the sticking plaster from which the backing layer or protective cover or both have been removed.

18. A dispenser according to claim 14 in which the dispenser is provided with a guide for directing movement of the layers separated from the sticking plaster into the second or third chambers in which the guide is located at or towards the exit of the channel.

19. A dispenser according to claim 18 in which the guide is in the form of a freely rotating roller located at or towards the end of the channel for directing the backing strip into the second chamber after being separated from the wound dressing or in the form of an end wall portion having a generally curved surface for directing the protective layer into the third chamber after having been separated from the wound dressing.

20. A dispenser according to claim 1 further comprising an operating subassembly having a retractable pin located axially along the lengthwise extending direction of the actuating lever for axial movement along the lengthwise extending axis of the lever between an extended position for engaging part of the mechanism for moving the movable member on a release position in which the pin is disengaged from the mechanism.

21. A dispenser according to claim 20 in which the operating subassembly includes a ratchet arrangement comprising a combination of teeth and grooves forming the mechanism for moving the movable member in which the end of the pin is engageable in selected ones of the grooves located between adjacent teeth when in the extended position and disengaged from the grooves when in the release position.

22. A dispenser according to claim 20 in which the operating subassembly further comprises a pair of intermeshed gear wheels arranged for rotation in opposite directions in response to movement of the actuating lever to move the wound dressing through the dispenser and to cause corresponding rotation of the rollers upon which the protective strip and backing strip are being wound during dispensing of the sticking plaster.

23. A dispenser according to claim 1 which is mountable on a solid substrate allowing operation of the dispenser by movement of the lever to dispense sticking plasters from the dispensing slot provided in the housing of the dispenser.

24. A dispenser according to claim 1 in which the backing strip is removed from the wound dressing simultaneously with the removal of the protective cover as the wound dressing is advanced through the dispenser.

25. A dispenser according to claim 1 in which the moveable member is capable of movement in one direction only to dispense the sticking plaster whereas the actuating element is capable of movement in two opposed directions.

26. A dispenser according to claim 1 in which the actuating element is movable between an engaging position and a release position in which the engaging position allows movement of the wound dressing and the release position allows movement of the actuating element whilst the wound dressing remains stationary.

27. A method of dispensing a single article from a multitude of similar articles using the dispenser of claim 1 in which each article includes at least one removable layer adhered to the article, said method comprising the steps of moving an actuating element to move a movable element to advance at least one of the multitude of similar articles through the dispenser so that the removable layer is separated from the article simultaneously with or prior to dispensing of the article from the dispenser.
